(19) United States
(12) Patent Application Publication
(10) Pub. No.: US 2015/0289860 A1
(43) Pub. Date: Oct. 15, 2015

(54) INTERCOSTAL DELIVERY SYSTEM AND METHODS THEREOF

(71) Applicant: NOVOGATE MEDICAL LTD., Yockneam Elite (IL)

(72) Inventors: Gil BOLOTIN, Zichron Yaakov (IL); Ziv BELSKY, Haifa (IL); Gilad Hizkiyahu, Givat Ela (IL)

(21) Appl. No.: 14/439,371

(22) PCT Filed: Nov. 3, 2013
(86) PCT No.: PCT/IL2013/050900

§ 371 Date: Apr. 29, 2015

Related U.S. Application Data
(60) Provisional application No. 61/722,142, filed on Nov. 3, 2012.

(51) Int. Cl. A61B 17/00; A61B 17/064; A61B 17/068; A61B 17/02

(52) U.S. Cl. CPC A61B 17/0057; A61B 17/00234; A61B 17/0218; A61B 17/064; A61B 17/068; A61B 2017/0237; A61B 2017/00243; A61B 2017/00579; A61B 2017/0061; A61B 2017/00623

(57) ABSTRACT

The invention discloses a delivery device for use in surgery on an organ in a living body. The delivery device is characterized by a proximal and a distal end inter-connected by a main longitudinal axis, the distal end comprising: (a) at least one anchoring device, comprising at least one tissue anchor; and (b) at least one pivoting support in communication with the anchoring device. The anchoring device can pivot freely around the pivoting support independent of the orientation of the pivoting support with respect to the organ, such that the anchoring device can be oriented substantially parallel to and conform to the surface of the organ with which the anchoring device comes into contact.

### FIELD OF THE INVENTION

The present invention generally pertains to a system and method for delivering a tissue anchoring device with a diameter larger than that of the intercostal space (about 10 mm) into the body by tilting at least a portion of the device during passage between the ribs.

### BACKGROUND OF THE INVENTION

With growing complexity of intra-cardiac procedures and the tendency to reduce the invasiveness of such procedures, ways of accessing the heart without much inconvenience to the patient and without much damage to organs are sought.

Specifically, transcatheter aortic valve replacement (TAVI) procedures use apical access. A hole is made at the base of a heart which is beating, through which the procedure is performed. The hole is closed at the end of the procedure using a special kind of suture. In order to place this suture, the patient's ribs have to be expanded, requiring anesthesia and causing residual pain and, potentially, other side effects.

Some closure devices are described in the art, such as the closure device in US patent application 20120150224, but it is unclear how they are to be delivered to the apex of the heart without expanding the ribs. PCT/IB2012/052033 discloses a ay to deliver the closure device described in 20120150224 but requires folding and unfolding that device, which is not simple to achieve.

The purpose of this invention is to describe a device that enables the delivery of a tissue anchoring device with a diameter larger than that of the intercostal space (about 10 mm) into the body by tilting at least a portion of the device during passage between the ribs.

### SUMMARY OF THE INVENTION

It is an object of the present invention to disclose a system and method for delivering a tissue anchoring device with a diameter larger than that of the intercostal space (about 10 mm) into the body by tilting at least a portion of the device during passage between the ribs.

It is another object of the present invention to disclose a delivery device for use in surgery on an organ in a living body, characterized by a proximal and a distal end interconnected by a main longitudinal axis, said distal end comprising:
a. at least one anchoring device, said anchoring device comprising at least one tissue anchor; and,
b. at least one pivoting support in communication with said anchoring device; said anchoring device is adapted to pivot around said pivoting support ;

wherein said anchoring device can pivot freely around said pivoting support independent of (a) the distance between said pivoting support and said organ; and, (b) the orientation of said pivoting support with respect to said organ, such that said anchoring device can be oriented substantially parallel to and conform to the surface of said organ with which said anchoring device comes into contact.

It is another object of the present invention to disclose the delivery device, wherein said anchoring device is adapted to pivot around said pivoting support in at least two orthogonal degrees of freedom.

It is another object of the present invention to disclose the delivery device, wherein said anchoring device comprises a plurality of tissue anchors; said tissue anchors characterized by a proximal end and a distal end interconnected by a main longitudinal axis, said proximal end attached to at least one surface of said anchoring device and said distal end adapted to anchor said anchoring device in said organ.

It is another object of the present invention to disclose the delivery device, wherein said plurality of tissue anchors extend from said at least one surface of said anchoring device.

It is another object of the present invention to disclose the delivery device, wherein said surface comprises a loop-like coil of closed periphery.

It is another object of the present invention to disclose the delivery device, wherein said surface has at least two configurations: (a) an open configuration and (b) a closed configuration.

It is another object of the present invention to disclose the delivery device, wherein said anchoring device is able to elastically deform from said open configuration to at least said closed configuration.

It is another object of the present invention to disclose the delivery device, wherein said anchoring device is adapted to be attachable to said organ's surface in said open configuration.

It is another object of the present invention to disclose the delivery device, wherein said open configuration defines a central opening within said anchoring device such that minimally invasive surgical manipulation is performable therethrough.

It is another object of the present invention to disclose the delivery device, wherein, in said open configuration, said surface is substantially planar.

It is another object of the present invention to disclose the delivery device, wherein, in said closed configuration, said anchoring device is adapted to close at least one of a gap, a hole and a lesion in said organ.

It is another object of the present invention to disclose the delivery device, wherein the longitudinal axes of said tissue anchors are substantially perpendicular to the plane of said surface.

It is another object of the present invention to disclose the delivery device, wherein the proximal ends of said plurality of tissue anchors are disposed in a closed loop configuration.

It is another object of the present invention to disclose the delivery device, wherein said surface is comprised of a flexible and resilient material.

It is another object of the present invention to disclose the delivery device, wherein at least one said tissue anchor comprises at least one retention member.

It is another object of the present invention to disclose the delivery device, wherein said retention member is at the distal end of said tissue anchor.

It is another object of the present invention to disclose the delivery device, wherein said retention member widens at least a portion of said tissue anchor.

It is another object of the present invention to disclose the delivery device, wherein the shape of said retention member is selected from a group consisting of: arrowhead-like, barb-like, hook-like and any combination thereof.

It is another object of the present invention to disclose the delivery device, wherein said tissue anchors are comprised of a stiff material.

It is another object of the present invention to disclose the delivery device, wherein at least a portion of said anchoring device comprises a metallic non-thrombogenic material.

It is another object of the present invention to disclose the delivery device, wherein at least a portion of said anchoring device is comprised of a metal selected from a group consisting of stainless steel, titanium, nitinol, and any combination thereof.

It is another object of the present invention to disclose the delivery device, wherein at least a portion of said anchoring device is comprised of a material selected from a group consisting of a shape memory material, a super-elastic material, and any combination thereof.

It is another object of the present invention to disclose the delivery device, wherein at least a portion of said anchoring device is coated with an inert plastic coating.

It is another object of the present invention to disclose the delivery device, wherein said inert plastic coating is nylon.

It is another object of the present invention to disclose the delivery device, wherein said anchoring device comprises a resorbable material.

It is another object of the present invention to disclose the delivery device, wherein said pivoting support is adapted to be pivotally attached to said distal end of said delivery device.

It is another object of the present invention to disclose the delivery device, wherein said pivoting support is adapted to be fixedly attached to said distal end of said delivery device.

It is another object of the present invention to disclose the delivery device, wherein said anchoring device has at least two orientations: (a) a first orientation substantially parallel to said main longitudinal axis; and (b) a second orientation substantially perpendicular to said main longitudinal axis.

It is another object of the present invention to disclose the delivery device, additionally comprising at least one first mechanism for changing the orientation of said anchoring device with respect to said main longitudinal axis in at least one degree of freedom.

It is another object of the present invention to disclose the delivery device, wherein at least one said first mechanism comprises a member of a group consisting of strings, rods, cables, and any combination thereof.

It is another object of the present invention to disclose the delivery device, wherein said degrees of freedom for reorientation of said anchoring device are selected from a group consisting of (a) rotation about an axis substantially perpendicular to said main longitudinal axis and substantially parallel to said pivoting supports, (b) rotation about an axis substantially perpendicular to said main longitudinal axis and substantially perpendicular to said pivoting supports, and (c) along said main longitudinal axis.

It is another object of the present invention to disclose the delivery device, additionally comprising a second mechanism attached to said pivoting support adapted to reciprocally move said pivoting support along said main longitudinal axis of said delivery device.

It is another object of the present invention to disclose the delivery device, wherein said anchoring device is reversibly detachable from said pivoting support.

It is another object of the present invention to disclose the delivery device, additionally comprising at least one arm adapted to connect at least one of said pivoting supports to said delivery device.

It is another object of the present invention to disclose the delivery device, wherein said connection comprises a member of a group consisting of a rotating connection, a pivoting connection, a sliding connection, a fixed connection and any combination thereof.

It is another object of the present invention to disclose the delivery device, wherein said device is characterized by a transport configuration and an operating configuration; said transport configuration is characterized by positioning said tissue anchors substantially within the perimeter of said device; said operating configuration such that said tissue anchors substantially extend outside the perimeter of said device.

It is another object of the present invention to disclose the delivery device, additionally comprising a third mechanism adapted to reversibly transform said anchoring device from said transport configuration to said operating configuration.

It is another object of the present invention to disclose the delivery device, wherein said third mechanism comprises a coil spring, said transport configuration characterized by said spring being in a compressed state, and said operating configuration characterized by said spring being in an expanded state.

It is another object of the present invention to disclose the delivery device, wherein, in said operating configuration, said spring applies pressure onto said anchoring device such that said tissue anchors are substantially outside the perimeter of said delivery device.

It is another object of the present invention to disclose the delivery device, wherein said spring is a conical spring.

It is another object of the present invention to disclose the delivery device, wherein said organ is selected from a group consisting of: the heart, a lung, an artery, a vein, the stomach, the small intestine, the large intestine, the colon, and the uterus.

It is another object of the present invention to disclose the delivery device, wherein said delivery device comprises a thoroughgoing bore such that minimally invasive surgical manipulation is performable therethrough.

It is another object of the present invention to disclose the delivery device, wherein said pivoting support comprises at least one member selected from a group consisting of a gimbal, a joint, a ball joint, a pin, a protrusion, an axle, a shaft, a spindle, a rod, a ball, a slot, a hole and any combination thereof.

It is another object of the present invention to disclose the delivery device, wherein the distal tip thereof is characterized by a blunt tip, said blunt tip adapted to pass between and separate pre-cut tissue, thereby enabling passage of said device therethrough.

It is another object of the present invention to disclose the delivery device, wherein said blunt tip prevents cutting of tissue by said device.

It is another object of the present invention to disclose the delivery device, wherein said blunt tip is comprised of two separable portions, each said separable portion characterized by a proximal and a distal end.

It is another object of the present invention to disclose the delivery device, wherein said proximal portions of said separable portions are movably attachable to said delivery device.

It is another object of the present invention to disclose the delivery device, wherein the distal ends of said separable portions are linkably contactable.

It is another object of the present invention to disclose the delivery device, wherein said distal ends are reversibly linked.

It is another object of the present invention to disclose the delivery device, wherein said link between said distal ends is selected from a group consisting of: a pin, a latch, and any connection thereof.

It is another object of the present invention to disclose the delivery device, wherein said blunt tip substantially retains its shape during passage through said pre-cut tissue.

It is another object of the present invention to disclose a method of performing minimally invasive surgery on an organ in a living body, comprising steps of:
a. providing a delivery device for use in surgery on an organ in a living body, characterized by a proximal and a distal end interconnected by a main longitudinal axis, said distal end comprising:
   i. at least one anchoring device, said anchoring device comprising at least one tissue anchor; and,
   ii. at least one pivoting support in communication with said anchoring device; said anchoring device is adapted to pivot around said pivoting support
b. inserting said delivery device into said body to approximate said organ;
c. pivoting said pivoting support, thereby substantially conforming said anchoring device to the surface of said organ
   wherein said step (c) of pivoting provides a free rotation around said pivoting support independent of (a) the distance between said pivoting support and said organ; and, (b) the orientation of said pivoting support with respect to said organ, such that said anchoring device can be oriented substantially parallel to the surface of said organ with which said anchoring device comes into contact.

It is another object of the present invention to disclose the method, additionally comprising steps of providing said anchoring device with a plurality of tissue anchors; said tissue anchors characterized by a proximal end and a distal end interconnected by a main longitudinal axis, said proximal end attached to at least one surface of said anchoring device and said distal end adapted to anchor said anchoring device in said organ.

It is another object of the present invention to disclose the method, additionally comprising steps of providing said surface comprising at least two configurations: (a) an open configuration and (b) a closed configuration.

It is another object of the present invention to disclose the method, wherein, in said open configuration, a central opening is provided in said anchoring device through which minimally invasive surgical manipulation can be performed.

It is another object of the present invention to disclose the method, additionally comprising steps of characterizing said device by a transport configuration and an operating configuration.

It is another object of the present invention to disclose the method, additionally comprising steps of characterizing said transport configuration by said pivoting support being positioned substantially parallel to said delivery device and, at such times as said anchoring device is present, said tissue anchors are positioned substantially within the perimeter of said device.

It is another object of the present invention to disclose the method, additionally comprising steps of characterizing said operating configuration by said pivoting support being positioned substantially perpendicular to the surface of said organ with which said anchoring device comes into contact, and, at such times as said anchoring device is present, said tissue anchors substantially extend outside the perimeter of said device.

It is another object of the present invention to disclose the method, additionally comprising steps of:
a. extending said anchoring device from said delivery device in said open position, thereby extending said tissue anchors to embed said anchoring device in said organ in said open position;
b. cutting into said organ; said cut being made through said anchoring device;
c. performing said minimally invasive operation via said cut;
d. detaching said anchoring device from said pivoting support; thereby releasing said anchoring device and allowing the same to elastically deform from said open configuration to said closed configuration and thereby to close said cut;
e. pivoting said pivoting support to said transport position, thereby substantially minimizing the thickness of said delivery device; and,
f. removing said delivery device from said living body via said insertion point.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates an embodiment of the delivery device;

FIG. 2 schematically illustrates an embodiment of the head of the device, showing the anchoring device;

FIG. 3 schematically illustrates the angles of an arrowhead;

FIG. 4A-C schematically illustrates pivoting of the anchoring device about the head of the delivery device; and

FIG. 5A-L schematically illustrates the steps involved in performing a minimally invasive operation using the delivery device.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for delivering a tissue anchoring device to a site with a living body.

The term ‘open configuration’ hereinafter refers to a configuration of the anchoring device such that, during operations, tools can be passed through it and into the heart or other organ being operated on.

The term ‘closed configuration’ hereinafter refers to a configuration of the anchoring device such that it can hold closed a gap, hole, lesion or cut into the heart or other organ.

The term ‘transport configuration’ hereinafter refers to a configuration of the delivery device such that the pivoting support and the anchoring device (if present) are substantially parallel to the main longitudinal axis of the device. The anchoring device, if present, and especially its tissue anchors, does not extend beyond the perimeter of the device. If the pivoting support has enclosing sides, the anchoring device and its tissue anchors is contained substantially within the pivoting support; the tissue anchors do not extend substantially beyond the sides of the pivoting support. In this manner, the tissue anchors are prevented from coming into contact with, and possibly damaging, tissue in the body during transport of the anchoring device through pre-cut tissue. In addition, at least during inward transport, a blunt tip is in position at the distal end of the head.

The term ‘operating configuration’ hereinafter refers to a configuration of the delivery device such that the pivoting support and the anchoring device are substantially perpendicular to the main longitudinal axis of the device. The anchoring device, and especially its tissue anchors, extend significantly beyond the perimeter of the device and the tissue anchors extend significantly beyond the sides of the pivoting support.

The term ‘holding force’ hereinafter refers to the force exerted by tissue anchors on the tissue that they are anchored to or embedded in. The holding force is exerted when a force perpendicular to the tissue is applied to the device and the tissue anchors. The holding force prevents the closure device of the present invention from being extracted from the tissue site by a perpendicular force that may be exerted on it.

The term ‘shape memory materials’, ‘shape memory polymers’ ‘shape memory alloys’, ‘nitinol’ and ‘super-elastic materials’ are used interchangeably to refer to materials capable of employing shape memory, where the materials can be deformed from an original, heat-stable configuration, to a second, heat-unstable configuration. An article is said to have shape memory when, upon the application of a trigger, for example heat, it can be caused to revert, or to attempt to revert, from its heat-unstable configuration to its heat-stable configuration so that it “remembers” its original heat-stable configuration or shape.

The term ‘approximately’ or ‘about’ hereinafter refers to within 20% of a value.

Note that 1N=1 gm-force*g, where g is the acceleration due to gravity, approximately 9.8 m s⁻². As used herein, 1N≈10 gm-force; the N values are for reference and the gm-force values are limiting.

By way of example, this description relates to the delivery of an anchoring device for apical procedures, but it may serve any device that has one dimension, height for example, that is less than the distance between relatively immobile body structures. For example, the distance between adjacent ribs in a human skeleton is typically up to about 10 mm. In order to pass something between the ribs without needing to expand the ribs and increase the distance between them, it is necessary that at least one dimension of the passing device be no larger than about 10 mm. Therefore, the exemplary device disclosed herein has a thickness less than about 10 mm and other dimensions that are up to about 40 mm each.

The intercostal delivery system disclosed herein comprises a pivoting support rotatable about a head, the head attached to a delivery device with a hollow throughgoing bore. The delivery device also serves as a handle, by means of which a surgeon can hold and manipulate the intercostal delivery system. The pivoting support, in preferred embodiments, is substantially in the form of five faces of a rectangular solid, with a first face being approximately square, having length of up to about 40 mm and width up to about 40 mm. The other four faces form the sides of the rectangular solid, surrounding the approximately square face. The depth of the sides is up to approximately 10 mm. The pivoting support has at least two positions relative to the handle, a first position wherein the pivoting support is substantially contained within the width and thickness of the handle, with the first face parallel to sides of the head, and a second position wherein the first face is substantially non-parallel to the pivoting support. In some embodiments, the pivoting support can rotate in a full circle relative to the head.

The device disclosed herein can be used for apical surgery on the heart. When the pivoting support is in its parallel position, the head and the pivoting support can pass easily through a slit in the chest wall and between the ribs to the heart. The pivoting support can then be rotated such that the first face is substantially parallel to the surface of the heart. The anchoring device can then be extended from the pivoting support so that the tissue anchors penetrate the heart and attach the anchoring device to the heart, as described hereinbelow, and surgical instruments, as described hereinbelow, can be passed through the device handle, through the pivoting support and through the anchoring device to reach the heart. An access port is made in the heart wall, e.g by using Seldinger's technique of expanding a needle hole to a 1 cm opening through the wall and surgical instruments are passed through the pivoting support, through the opening and into the heart. After completion of the surgery, the anchoring device can be detached from the pivoting support, as described hereinbelow, the pivoting support returned to its first position substantially parallel to the head, and the device is removed from the body through the slit in the chest wall. After detachment from the pivoting support, the attachment mechanism, as described hereinbelow, causes closure of the opening in the heart wall, so that no sutures are needed to close the opening in the heart.

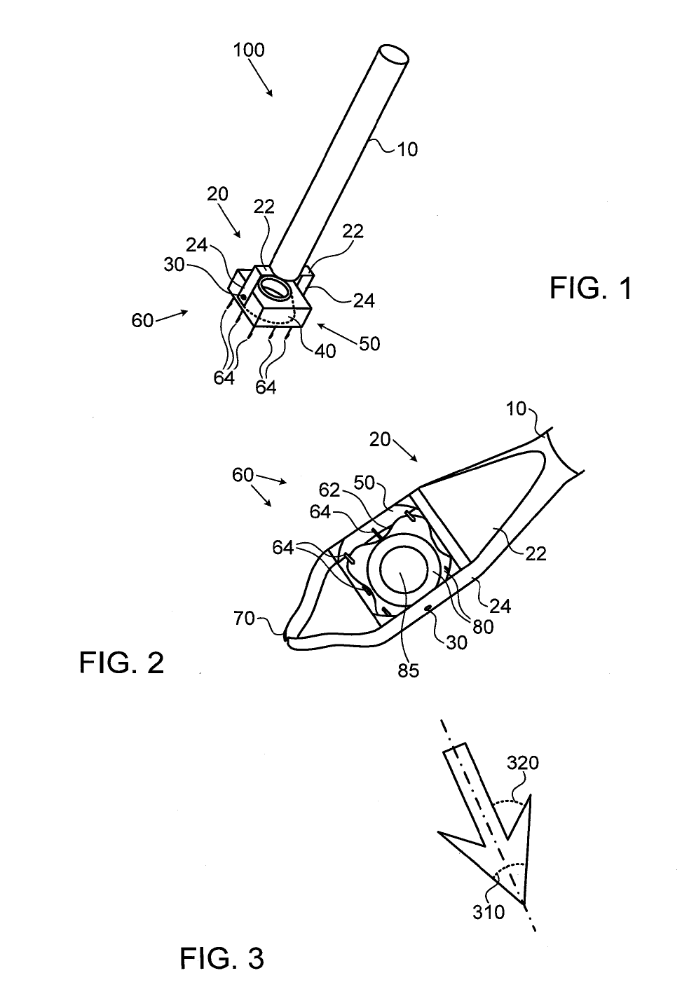

FIG. 1 illustrates an embodiment of an intercostal delivery system 100. An elongated, essentially tube-like handle 10 is used to control and manipulate the intercostal delivery system 100.

A U shaped head 20 with its base 22 and arms 24 is affixed by its base 22 to the distal end of the handle 10. The U-shaped head 20 is wide enough to allow pivoting support 50 and anchoring device 60 to reside between the two arms 24 of the U shaped head 20. Near the distal ends of the arms 24, there are pivot points 30. Pivoting support 50 is connected to arms 20 via pivot points 30, and can rotate about an axis through pivot points 30. The pivot can comprises a pivot pin, at least one protrusion on at least one arm 24, at least one protrusion on at least one side of the pivoting support 50, and any combination thereof. Pivot pins can be attached to an arm 24, a side of the pivoting support 50, rotate freely within a hole in an arm 24, rotate freely within a side of the pivoting support 50, and any combination thereof that allows the pivoting support to rotate with respect to the U-shaped head 20 about the pivot points 30. The pivot can also be a ball joint, with a sphere-like protrusion movable within a concave surface (partial sphere-like crater). This type of connection allows movement in several directions around the pivot point, not only about the pivot axis.

In some embodiments, the pivoting support 60 can be fixedly attached to the distal end of the delivery device 100.

Also shown in FIG. 1 are the tissue anchors 64 of the anchoring device 60, discussed hereinbelow, which are adapted to attach the anchoring device 60 to the heart, and the compression spring 40, discussed hereinbelow, which, in preferred embodiments, pushes the anchoring device 60 at least partly out of the pivoting support 50. In FIG. 1, the compression spring 40 is shown in its expanded position, with the anchoring device 60 substantially outside of the pivoting support 50. The device is in the position shown in FIG. 1, with the spring 40 expanded and the anchoring device 60 substantially outside of the pivoting support 50, during the period when the anchoring device 60 is implanted in a heart.

The anchoring device 60 provides a device for tissue closure configured to have an approximately circular arrangement that provides a stable and efficient radial closing force about a tissue opening. The anchoring device 60 can also be retained in an open configuration adapted to allow passage of tools therethrough.

In preferred embodiments, compression spring 40 comprises a conical spring, with successive coils of the spring fitting inside each other so that a fully-compressed spring 40 is substantially the thickness of a single coil thereof.

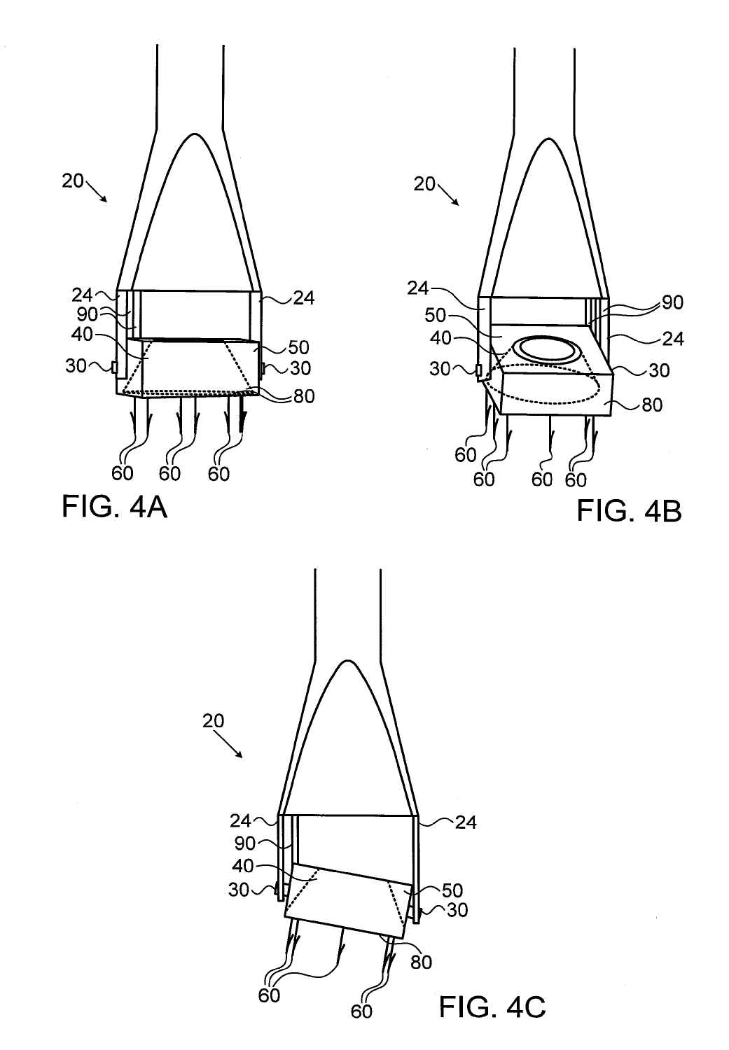

FIG. 2 illustrates an embodiment of the U-shaped head 20 and the distal end of the handle 10. The pivoting support 50 is shown in a position such that it is substantially within the U-shaped head 20; in this position, a gap just large enough to allow passage of the U-shaped head 20 will also allow passage of the pivoting support 50 and everything within it.

In this embodiment, the base 22 of the U-shaped head 20 has been elongated and its sides sloped, allowing the width of the device to transition smoothly from its width across the arms 24 (somewhat greater than the width of the pivoting support 50) to its width across the handle 10 (the sideways diameter of the handle 10). In this embodiment, the thickness of the base 22 also transitions smoothly from a thickness substantially the thickness of the pivoting support 50 to a thickness substantially the thickness-direction diameter of the handle 10.

Inside the pivoting support 50 is anchoring device 60, comprising a loop-like surface 62 and tissue anchors 64, held in place by spring compression cap 80.

Compression cap 80 is releasably attached to device pivoting support 50. The release mechanism can be a removable pin linking compression cap 80 to device pivoting support 50, a movable arm, a releasable latch, or any method know in the art where release can be effectuated by a pull or push triggerable from the proximal end of the handle 10. Upon release, in preferred embodiments, compression cap 80 is slidably connected to device pivoting support 50.

In some embodiments, releasing the compression cap 80 from the device pivoting support 50 causes the compression cap 80 to be pushed at least partially out of device pivoting support 50. In other embodiments, activating the release mechanism enables movement of the compression cap 80 relative to the pivoting support 50, but the compression cap 80 is pushed at least partially out of the pivoting support 50 by a second mechanism activatable by a second control near the proximal end of the handle 10. The second control can be a pressable button, a movable lever, or any other means known in the art of activating a pushing mechanism.

The compression cap 80 is used to compress the spring (40, not shown) so that it takes up a minimum of device pivoting support 50 (such as approximately 1-2 mm).

It is to be noted that the compression cap 80 has a hole 85 approximately in its center such that, during operations, tools passed through the handle 10 and head 20 of the device can pass through the compression cap 80 and into the heart.

In preferred embodiments, the diameter of the hole is in the range of about 16 mm to about 25 mm.

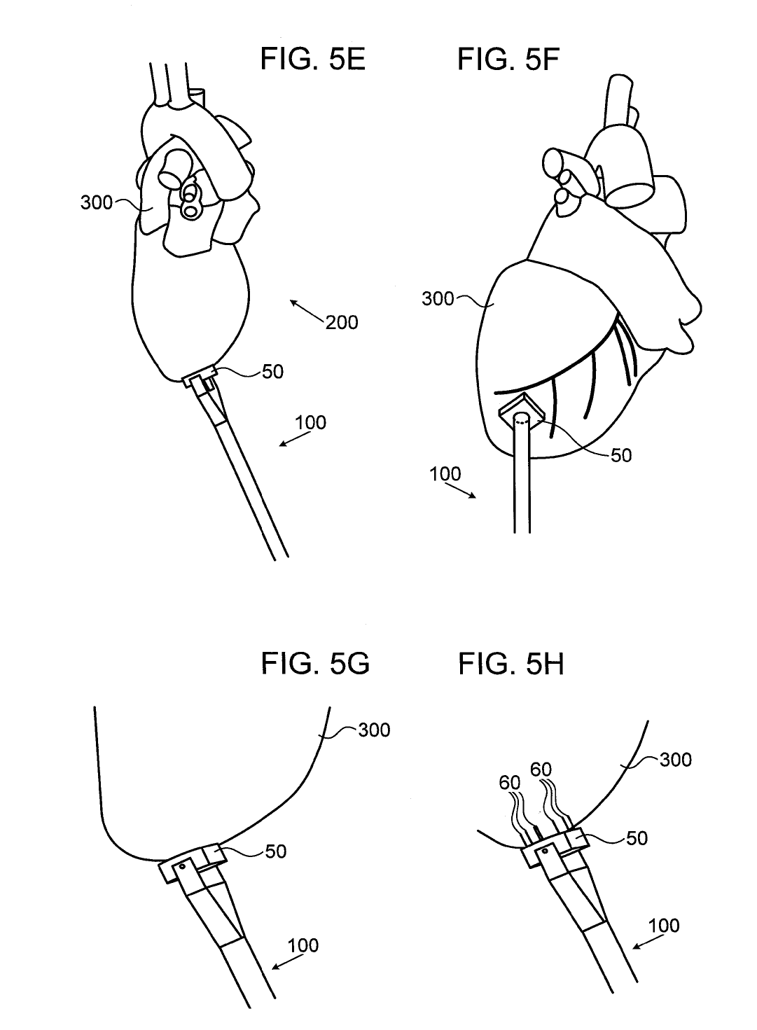

In preferred embodiments, compression cap 80 also holds loop-like anchoring surface 62 in its open configuration, as shown in FIG. 2; release of anchoring device 60 from compression cap 80 or from a separate mechanism holding anchoring device 60 in the open configuration allows anchoring device 60 to assume its closed configuration (see FIGS. 4K-L hereinbelow).

In preferred embodiments, the tissue anchors 64 have a length from about 5 mm to about 7 mm.

In preferred embodiments, the force needed to pull out a tissue anchor 64 embedded in tissue (the holding force) is between about 60 gm-force and about 180 g-force (about 0.6N-about 1.8N).

In preferred embodiments, the force needed to embed a tissue anchor 64 in tissue (the embedding force) is less than about 60 gm-force (about 0.6N).

FIG. 1 and FIG. 4A-C hereinbelow show the pivoting support 50 and the anchoring device 60 in the operating configuration, with the pivoting support 50 and the anchoring device 60 substantially perpendicular to the main longitudinal axis of the head 20 (and the device 100) and with the tissue anchors 64 extending substantially outside pivoting support 50 and substantially beyond the perimeter of the head 20. The blunt tip 70 has been retracted and is not shown.

FIG. 2 shows the pivoting support 50 and the anchoring device 60 in the transport configuration, with the pivoting support 50 and the anchoring device 60 parallel to the main longitudinal axis of the head 20 (and the device 100). The anchoring device 60 is withdrawn substantially within the pivoting support 50, so that no part of it, and especially none of the tissue anchors 64, extends beyond the perimeter of the device. In this manner, the tissue anchors are prevented from coming into contact with, and possibly damaging, tissue in the body during transport of the anchoring device through pre-cut tissue.

For inward transport, the blunt tip 70 is in position at the distal end of the head 20.

It should be noted that, in both the transport configuration and the operating configuration of the device, the anchoring device 60 is in the open configuration, since, when the anchoring device 60 is attached to the pivoting support 50, compression cap 80 holds the anchoring device 60 in the open position. The anchoring device 60 will only assume its closed configuration after it has been detached from the compression cap 80 in the delivery device 100 .

The anchoring device 60 provides a tissue closure device that can be anchored in tissue with a plurality of anchors 64, wherein closure is achieved without folding the tissue anchors within the tissue to achieve closure, while providing sufficient radial force to close a tissue site.

In preferred embodiments, the force needed to close the opening in the heart, the closing force, is more than about 350 gm-force (about 3.5N).

In preferred embodiments, there is a closing force configured to close the tissue site opening by exerting a radial force on the tissue in the range of from about 300 gm-force to about 500 gm-force (about 3N to about 5N).

In preferred embodiments, there is a closing force configured to close the tissue site opening by exerting a radial force on the tissue in the range of from about 200 gm-force to about 350 gm-force (about 2N to about 3.5N).

In embodiments comprising metallic material, the metallic material is non-thrombogenic.

In some embodiments, the anchoring device 60 is comprised of, for non-limiting example, super-elastic materials (‘SE’), shape memory polymers, shape memory alloys (‘SMA’), plastics, alloys and materials capable of super-elastic and/or memory shape properties and having a plurality of stable configurations. Such materials, alloys and polymers can comprise, but are not limited to, Ni—Ti, Ni—Ti alloys, nitinol, Cu-based alloys, Cu—Zn—Al, Au—Cd, Ni—Al, stainless steel 316, polymers, BeCu alloy, CoCr alloy, Ag—Cd, Au—Cd, Cu—Al—Ni, Cu—Sn, Cu—Zn, Cu—Zn—Si, Cu—Zn—Sn, Fe—Pt, Mn—Cu, Fe—Mn—Si, Pt alloys, Co—Ni—Al, Co—Ni—Ga, Ni—Fe—Ga, Ti—Pd, Ni—Ti—Nb, Ni—Mn—Ga, other similar materials that are known in the art, and any combination thereof.

In preferred embodiments comprising shape memory materials, the shape memory materials have a transformation temperature in at least one of the following ranges: between about −18° C. and about 25° C. and preferably between about 10° C. and 25° C.

In some embodiments, transformation of a shape memory material can be by a means selected from a group consisting of: temperature change, electromagnetic field, exposure to a predetermined chemical, and any combination thereof.

In preferred embodiments, the surface 62 comprises a resilient material with at least one of spring-like or elastic properties. In the open configuration, the resilient material is in an expanded or stretched configuration; when released from the open configuration, the resilient material returns to its relaxed, unexpanded or unstretched configuration; if embedded in tissue, it thereby exerts a closing force on the tissue.

In embodiments comprising non-resorbable materials, the anchoring device 60 is preferably coated, at least partially, with an inert plastic coating. In preferred variants of this embodiment, the inert plastic coating is comprised of nylon, although any inert plastic known in the art can be used.

In preferred embodiments, the tissue anchors 62 are comprised of a stiff material.

Typically, the material comprising the surface 62 of the anchoring device 60 has thickness in at least one of the following ranges: from about 0.25 mm to about 2.0 mm, preferably from about 0.4 mm to about 1.2 mm, and most preferably about 0.6 mm. its height is from about 0.25 mm to about 2.0 mm, preferably from about 0.4 mm to about 1.2 mm, and most preferably about 0.8 mm.

Typically, the material comprising the tissue anchors 64 of the anchoring device 60 have thicknesses of about 0.6 mm.

Typically, the tissue anchors 64 have length in the range of about 5 mm to about 7 mm, preferably about 6 mm.

In some embodiments, the tissue anchors 64 are of substantially the same thickness throughout. In preferred embodiments, at least one of the tissue anchors 64 comprises a retention means, preferably at the distal end of the tissue anchor 64. Typically, the retention means is a barb-like, hook-like or arrowhead-like configuration on the tissue anchor 64. The retention means enables more secure anchoring and/or embedding of the anchoring device 60 within a the tissue, meaning the retention means significantly increases the holding force without significantly increasing the force required to insert the tissue anchor into the tissue. The retention members shown in FIG. 2 are arrowhead-like, whereas the retention members shown in FIG. 1 are barb-like. A hook-like retention member typically includes a change in the direction of the anchor material without a change in the thickness of the anchor material, or an extension of the anchor material sideward but not backward.

In some embodiments, the tissue anchors 64 comprise at least one skyved side projection that can extend from the sides of the body of the tissue anchor 64. In preferred embodiments, the skyved projection provides for easy insertion of the tissue anchor 64 into a tissue site, but prevents removal of the tissue anchor 64 from the tissue site, thereby providing a holding force for the tissue anchor and preventing extraction from the tissue site.

In some embodiments, the skyved projections are fixed. In other embodiments, the skyved projections extend from the body of the anchor when a perpendicular force is exerted on the anchor. In such embodiments, the skyved tissue anchors can comprise, for non-limiting example, SMA or SA, including NiTi or similar alloys.

Arrowhead-like configurations can have a width in the range from about 0.8 mm to about 3 mm, preferably about 1.5 mm.

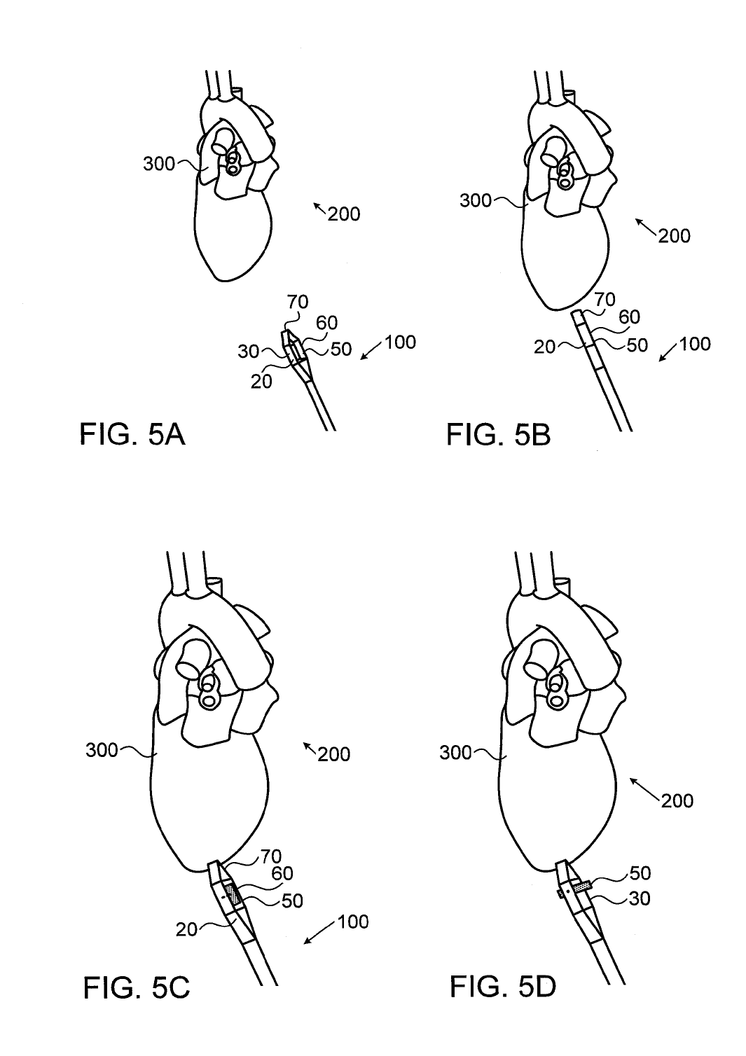

In reference to FIG. 3, embodiments with an arrowhead-like configuration the tip comprises an external angle 310 in at least one of the following ranges: from about 45 degrees to about 15 degrees, preferably from about 25 to about 35, and most preferably about 30 degrees.

In embodiments with an arrowhead-like configuration, the arrowhead can have an internal angle 320 in at least one of the following ranges: from about 40 degrees to about 75 degrees, preferably from about 45 degrees to about 65 degrees, more preferably from about 50 degrees to about 60 degrees, and most preferably about 56 degrees.

In embodiments with arrowhead-like configurations, the arrowhead can be thicker than the material to which it is attached. In preferred embodiments, the thickness of the arrowhead is less than about 0.15 mm, but no less than the thickness of the main body of the tissue anchor 64.

In FIG. 2, the compression spring (not shown) is in its compressed configuration, so that the anchoring device 60 is retracted within the pivoting support 50.

FIG. 2 further illustrates elongated, blunt tip 70, which comprises two halves, one adjacent each side of the U-shaped head 20. The two halves are releasably linked at their distal end, with the releasing mechanism comprising a pin, a latch, or any mechanism known in the art that can allow easy separation of linkable portions. In preferred embodiments, the releasing mechanism can be activated by, for non-limiting example, pulling a string which passes through at least as portion of the handle 10. After release of the mechanism so that the distal tips can separate, the two halves of elongated blunt tip 70 can be pulled back towards the U-shaped head 20, for example by pulling on pull strings, where the strings' distal ends are attached to the proximal ends of the halves of the elongated blunt tip 70 and the strings' proximal ends extend at least partly through the body of handle 10. In preferred embodiments, the same mechanism both enables separation of the distal tips of the halves of elongated blunt tip 70 and retraction of the halves of elongated blunt tip 70 towards the U-shaped head 20.

In some embodiments, the releasable linkage is a reversible linkage.

Elongated, blunt tip 70 is used to allow the delivery device 10 to penetrate between adjacent ribs through tissue that has been pre-cut, as will be explained below.

In preferred embodiments, when fully retracted, the distal tips of the two halves of elongated, blunt tip 70 are adjacent to the pivot point 30 on U-shaped head 20. In other embodiments, when fully retracted, the distal tips of the two halves of elongated, blunt tip 70 are adjacent to another point along the side of arms 24. In all embodiments, when fully retracted, the two halves of elongated, blunt tip 70 are substantially parallel to and are adjacent to the sides of head 20. In preferred embodiments, the two halves of elongated, blunt tip 70 do not extend substantially onto handle 10.

Elongated, blunt tip 70 is sufficiently blunt such that it is incapable of further cutting tissue during its passage through pre-cut tissue. It can maintain its shape during passage through pre-cut tissue and will not deform by more than approximately 3 mm even when a force of between approximately 100 gm-force and approximately 400 gm-force (approximately 1N-approximately 4N) is applied to its tip.

FIG. 4A-C illustrates the an embodiment of the head 20 and pivoting support 50, with the compression spring 40 in its expanded configuration and the compression cap 80 and anchoring device 60 in their extended position. Note that, in FIG. 4A-C, of anchoring device 60, only the tissue anchors 64 are visible. FIG. 4A-C illustrates an embodiment of a mechanism for changing the orientation of the pivoting support 50 and its contents, compression spring 40, compression cap 80 and anchoring device 60, with respect to the head 20. In this embodiment, the mechanism comprises pull-strings 90. By means of the pull-strings 90, steering rods, or other reorientation mechanisms such as are known in the art, the surgeon can orient the pivoting support 50 and its contents 40, 80, 60 so that the anchoring device 60 is parallel to the surface of the heart or other organ before the anchoring device 60 is extended from the device, thereby ensuring proper alignment of the anchoring device 60 with the heart,

In preferred embodiments, the position of at least one of the pivot points 30 can be altered, such that pivoting support 50 can be titled with respect to the head 20. Therefore, in preferred embodiments, the pivoting support 50 has at least two degrees of freedom of motion, namely, rotation about two axes, the two axes being perpendicular to the main longitudinal axis of the arms 24 and perpendicular to each other.

In FIG. 4A, the pivoting support 50 is shown perpendicular to the arms 24, In FIG. 4B, the pivoting support has been rotated about the pivot point 30, but the pivot points have not been moved. In FIG. 4C, the pivot point on the left in the figure has been moved upward toward proximal end of left-hand arm 24, tilting the pivoting support toward the right in the figure.

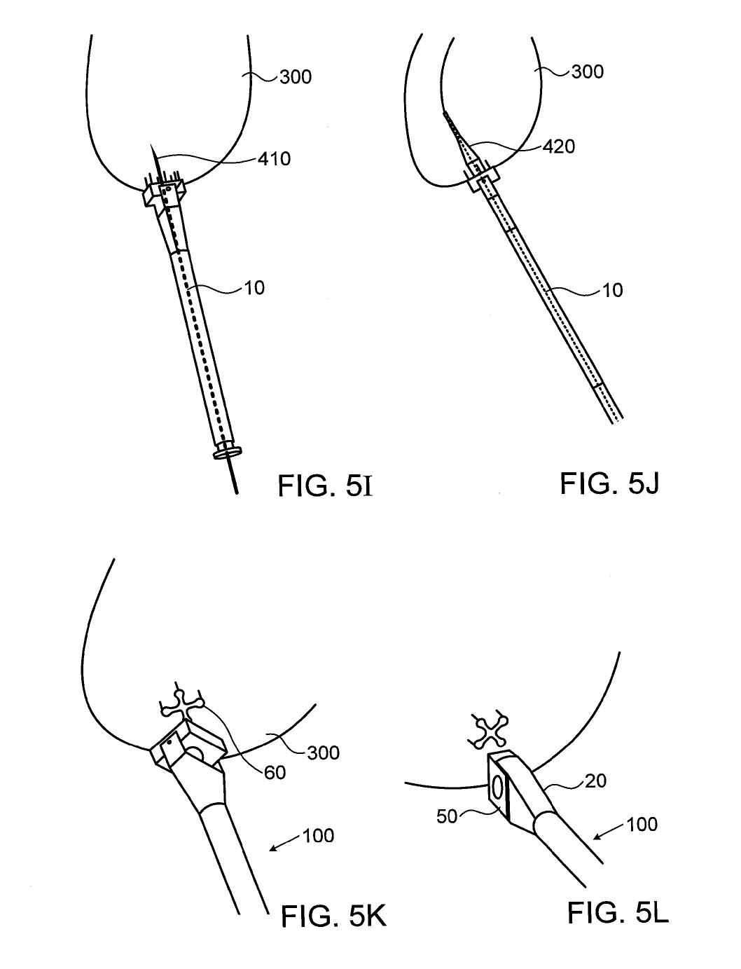

By way of example, FIG. 5A-L will illustrate the use of the delivery tool in a transcatheter aortic valve replacement (TAVI) procedure in which apical closure is to be effected using anchoring device 60.

FIG. 5A illustrates an embodiment of the intercostal delivery system 100 in its entirety, with the anchoring device 60 mounted within, prepared for use. The elongated blunt tip 70 is extended so that the halves meet and are held in place at the distal end of the delivery device. Device pivoting support 50 is rotated so that it is parallel to the U-shaped head 20 so that the height of the front part of the delivery device is minimal. The heart 300 and the chest wall and ribs 200 are shown schematically.

FIG. 5B illustrates the intercostal delivery system 100 during its insertion between the ribs 200 through an incision made previously with a scalpel. Due to the elongated blunt tip 70 the delivery device can push through the tissue, even though the gap in the tissue is not being held open. The head 20 is parallel to the ribs, the pivoting support 50 is parallel to the head 20, and the anchoring device 60 is fully retracted within the pivoting support 50.

In FIG. 5C, the head 20 of the intercostal delivery system 100 is in the body, with the elongated blunt tip 70 adjacent to the heart. The pivoting support 50 is parallel to the head 20, and the anchoring device 60 is fully retracted within the pivoting support 50. The head 20 need no longer be parallel to the ribs.

In FIG. 5D, the device pivoting support 50 has been rotated by 90 degrees around pivot points 30, as explained above.

In FIGS. 5E-G, the halves of the elongated blunt tip 70 (not shown) have been separated and retracted as explained above and the device pivoting support 50 advanced until it is exactly adjacent the heart. FIG. 5E shows the intercostal delivery system 100 and the heart from the side, at the same angle as FIGS. 4A-D, while, in FIG. 5F, the heart 300 and intercostal delivery system 100 are shown looking upward toward the apical tip of the heart and, in FIG. 5G, the heart 300 and intercostal delivery system 100 are shown from the side, at a different angle from FIGS. 4A-E. In FIGS. 5F-G, the schematic 200 of the chest wall and ribs is not shown.

At this point in the procedure, the user (physician) can allow spring 40 to decompress,by releasing it from the cap 80 as explained above. The release from cap 80 allows spring 40 to resume its extended form and push the anchoring device 60 from within the device pivoting support 50 and into the heart tissue. FIG. 5H, illustrates the situation after release of spring 40, with intercostal delivery system 100 adjacent to the heart, and the anchoring device 60 extended from the pivoting support 50 and attached to the heart 200.

FIG. 5I illustrates, inserted through handle 10 into the heart 300, a needle 410 to create the initial hole in the heart, as part of Seldinger's technique to create a hole in the heart, as a non-limiting example of a tool typically used in the exemplary TAVI surgery on the heart.

FIG. 5J illustrates another such tool, the Ascendra™ TAVI delivery sheath 420, inserted through handle 10 into the heart 300.

FIG. 5K-L illustrate the ending phase of the procedure, after the valve has been replaced and the surgical tools removed.

FIG. 5K illustrates the intercostal delivery system 100 after it has been separated from the anchoring device 60. The anchoring device 60 has assumed its closed configuration, thereby closing the hole in the heart 300.

FIG. 5L shows the intercostal delivery device 100 in transport configuration, such that it can be removed from the body. The device pivoting support 50 has been rotated into a position parallel to the U-shaped head 20 so that the head 20 and pivoting support 50 can pass between the ribs and out of the body. In this embodiment, during outward transport, the blunt tip 70 (not shown) remains in its retracted position by the sides of the head 20. In other embodiments, the blunt tip 70 (not shown) is returned to the distal end of the head 20 prior to removal of the device 100 from the body.

In preferred embodiments, the anchoring device 60 is made of resorbable materials and is adapted to remain embedded in the heart until it is disposed of by the body's natural processes.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A delivery device for use in surgery on an organ in a living body, characterized by a proximal and a distal end interconnected by a main longitudinal axis, said distal end comprising:
a. at least one anchoring device, said anchoring device comprising at least one tissue anchor; and,
b. at least one pivoting support in communication with said anchoring device; said anchoring device is adapted to pivot around said pivoting support;
wherein said anchoring device can pivot freely around said pivoting support independent of (a) the distance between said pivoting support and said organ; and, (b) the orientation of said pivoting support with respect to said organ, such that said anchoring device can be oriented substantially parallel to and conform to the surface of said organ with which said anchoring device comes into contact.

2. The delivery device of claim 1, wherein said anchoring device is adapted to pivot around said pivoting support in at least two orthogonal degrees of freedom, said degrees of freedom for reorientation of said anchoring device selected from a group consisting of (a) rotation about an axis substantially perpendicular to said main longitudinal axis and substantially parallel to said pivoting supports, (b) rotation about an axis substantially perpendicular to said main longitudinal axis and substantially perpendicular to said pivoting supports, and (c) along said main longitudinal axis.

3. The delivery device of claim 1, wherein at least one of the following is being held true:
a. the longitudinal axes of said at least one tissue anchor is substantially perpendicular to the plane of said surface

4. The delivery device of claim 1, wherein at least one of the following is being held true:
a. said anchoring device comprises at least one surface;
b. said surface comprises a loop-like coil of closed periphery;
c. said surface has at least two configurations: (i) an open configuration and (ii) a closed configuration;
d. said anchoring device is able to elastically deform from said open configuration to said closed configuration;
e. said anchoring device is adapted to be attachable to said organ's surface in said open configuration;
f. said open configuration defines a central opening within said anchoring device such that minimally invasive surgical manipulation is performable therethrough;
g. in said open configuration, said surface is substantially planar;
h. in said closed configuration, said anchoring device is adapted to close at least one of a gap, a hole and a lesion in said organ
i. said at least one tissue anchor is characterized by a proximal end and a distal end interconnected by a main longitudinal axis, said proximal end attached to at least one surface of said anchoring device and said distal end adapted to anchor said anchoring device in said organ;
j. said at least one tissue anchor extends from said at least one surface of said anchoring device;
k. at least one said tissue anchor comprises at least one retention member;
l. said retention member is at the distal end of said tissue anchor;
m. said retention member widens at least a portion of said tissue anchor;
n. the shape of said retention member is selected from a group consisting of: arrowhead-like, barb-like, hook-like and any combination thereof;
o. said anchoring device comprises a plurality of tissue anchors;
p. the proximal ends of said plurality of tissue anchors are disposed in a closed loop configuration;

5. The delivery device of claim 1, wherein said pivoting support is adapted to be either pivotally or fixedly attached to said distal end of said delivery device.

6. The delivery device of claim 1, wherein said anchoring device has at least two orientations: (a) a first orientation substantially parallel to said main longitudinal axis; and (b) a second orientation substantially perpendicular to said main longitudinal axis.

7. The delivery device of claim 1, additionally comprising at least one first mechanism for changing the orientation of said anchoring device with respect to said main longitudinal axis in at least one degree of freedom; wherein at least one said first mechanism comprises a member of a group consisting of strings, rods, cables, and any combination thereof.

8. The delivery device of claim 1, additionally comprising a second mechanism attached to said pivoting support adapted to reciprocally move said pivoting support along said main longitudinal axis of said delivery device.

9. The delivery device of claim 1, wherein said anchoring device is reversibly detachable from said pivoting support.

10. The device of claim 1, additionally comprising at least one arm adapted to connect at least one of said pivoting supports to said delivery device; wherein said connection comprises a member of a group consisting of a rotating connection, a pivoting connection, a sliding connection, a fixed connection and any combination thereof.

11. The device of claim 1, wherein said device is characterized by a transport configuration and an operating configuration; said transport configuration is characterized by positioning said tissue anchors substantially within the perimeter of said device; said operating configuration such that said tissue anchors substantially extend outside the perimeter of said device.

12. The device of claim 1, additionally comprising a third mechanism adapted to reversibly transform said anchoring device from said transport configuration to said operating configuration.

13. The device of claim 12, wherein said third mechanism comprises a coil spring, said transport configuration characterized by said spring being in a compressed state, and said operating configuration characterized by said spring being in an expanded state; wherein, in said operating configuration, said spring applies pressure onto said anchoring device such that said tissue anchors are substantially outside the perimeter of said delivery device.

14. The device of claim 1, wherein said organ is selected from a group consisting of: the heart, a lung, an artery, a vein, the stomach, the small intestine, the large intestine, the colon, and the uterus.

15. The device of claim 1, wherein said delivery device comprises a thoroughgoing bore such that minimally invasive surgical manipulation is performable therethrough.

16. The device of claim 1, wherein said pivoting support comprises at least one member selected from a group consisting of a gimbal, a joint, a ball joint, a pin, a protrusion, an axle, a shaft, a spindle, a rod, a ball, a slot, a hole and any combination thereof.

17. The device of claim 1, wherein said distal tip thereof is characterized by a blunt tip, said blunt tip adapted to pass between and separate pre-cut tissue, thereby enabling passage of said device therethrough; wherein said blunt tip prevents cutting of tissue by said device.

18. The device of claim 17, wherein said blunt tip is comprised of two separable portions, each said separable portion characterized by a proximal and a distal end; said proximal portions of said separable portions are movably attachable to said delivery device and the distal ends of said separable portions are linkably contactable, said link between said distal ends being selected from a group consisting of: a pin, a latch, and any combination thereof.

19. The device of claim 18, wherein said blunt tip substantially retains its shape during passage through said pre-cut tissue.

20. The device of claim 4, wherein the radial force applied on said organ by said anchoring device in said closed configuration is in the range of about 2N to about 5N, preferably about 2N to about 3.5N.

21. A method of performing minimally invasive surgery on an organ in a living body, comprising steps of:
a. providing a delivery device for use in surgery on an organ in a living body, characterized by a proximal and a distal end interconnected by a main longitudinal axis, said distal end comprising:
i. at least one anchoring device, said anchoring device comprising at least one tissue anchor; and,
ii. at least one pivoting support in communication with said anchoring device; said anchoring device is adapted to pivot around said pivoting support;
b. inserting said delivery device into said body to approximate said organ;
c. pivoting said pivoting support, thereby substantially conforming said anchoring device to the surface of said organ;
wherein said step (c) of pivoting provides a free rotation around said pivoting support independent of (a) the distance between said pivoting support and said organ; and, (b) the orientation of said pivoting support with respect to said organ, such that said anchoring device can be oriented substantially parallel to the surface of said organ with which said anchoring device comes into contact.
